(19) United States
(12) Patent Application Publication
(10) Pub. No.: US 2026/0212157 A1
(43) Pub. Date: Jul. 23, 2026

(54) OPTIMIZED CLASS SPECIFIC EMBEDDING GENERATOR AND MULTI-BRANCH DECODER DEEP NETWORK FOR CARDIAC STRUCTURE SEGMENTATION

(71) Applicant: Ashish GHOSH, Kolkata (IN)

(72) Inventors: Ashish GHOSH, Kolkata (IN); Susmita GHOSH, Kolkata (IN); Rahul ROY, Hyderabad (IN)

(21) Appl. No.: 19/026,884

(22) Filed: Jan. 17, 2025

(51) Int. Cl. G06N 3/0455; A61B 8/00; A61B 8/08; G06N 3/096; G06V 10/774

(52) U.S. Cl. CPC G06N 3/0455; A61B 8/0883; A61B 8/5215; G06N 3/096; G06V 10/774

(57) ABSTRACT

The invention provides an optimized class specific embedding generator and multi-branch decoder deep network for cardiac structure segmentation. An encoder network receives labeled and unlabeled echocardiogram images from a training dataset. The encoder extracts image features, maps them to class specific features, and computes prototypes from labeled embeddings. A splitter layer receives encoded features subtracted from the prototype and transforms them into class specific embeddings. The splitter layer weights are learned during encoder training using a Matryoshka classifier. A decoder network with multiple decoders receives the class specific embeddings as input and decodes them to reconstruct segmentation maps of cardiac structures. This approach enables effective segmentation with limited annotated data by leveraging differential features learned from exemplar embeddings and framing reconstruction as a multitask learning problem.

### FIELD OF INVENTION

The present invention generally relates to the fields of cardiac structure imaging technology, more particularly related to a multi-branch encoder decoder deep network for cardiac structure segmentation in a teacher student framework to handle limited availability of annotated data.

### BACKGROUND OF THE INVENTION

Health is an important factor in life of every human and taking care of physical body is good for mental health of an individual. Nowadays many people suffering from heart related disorders or diseases such as heart attacks, arrhythmia, valvular malfunction, etc. The human heart is a four-chambered muscular organ, shaped and sized roughly like a man's closed first with two-thirds of the mass to the left of midline. Heart disease treatment depends on the cause and type of heart damage. There are some commonly used medical tests or methods to diagnose heart health condition such as blood tests, electrocardiogram, exercise stress test, Echocardiogram, etc.

Echocardiogram test, also known as cardiac ultrasound utilizes sound waves and echoes thereof to monitor heart rhythm. The visual cardiac images, also known as cardiovascular imaging are generated during the test are utilized to check cardiac structure and diagnose different conditions of the heart including cardiomyopathy and valve disease. Numerous strategies have been developed to segment cardiac structures from echocardiographic images. Various techniques and methods are available for automated echo image processing such as—Machine Learning techniques for echo image quality assessment, view classification, cardiac region segmentation, and disease diagnosis. Initially, segmentation methods relied on explicit features such as intensity, spectral, and texture characteristics of the echocardiographic images.

However, these methods struggled with the complexities of echocardiographic images that contain various degradations. Combining these features often resulted in redundant feature spaces, increasing the complexity of the segmentation model. In recent years, deep learning techniques have emerged as powerful tools for segmenting cardiac structures. The introduction of the U-Net architecture marked a significant advance in this domain. Subsequently, various adaptations of the U-Net model have been proposed to address the segmentation challenges posed by diverse image degradations. Nevertheless, the U-Net model has a notable drawback as it demands a substantial number of annotated samples, a scarcity observed in the case of cardiac ultrasound images. Researchers have tackled this limitation by resorting to two main approaches, sample augmentation and leveraging pre-trained networks. Sample augmentation involves introducing alterations, such as affine transformations or noise, into training samples. However, this form of enhancement often does not provide substantial geometric information, leading to a limited improvement in segmentation performance. A more promising avenue involves generating synthetic images based on known geometric priors. This approach has shown considerable potential to improve model training performance for segmenting structures.

By incorporating synthetic images that align with geometric expectations, researchers have achieved promising results in improving segmentation accuracy. Another avenue pursued to address the issue of limited data involved the utilization of pre-trained networks. In this approach, researchers delved into domain transfer and zero-shot learning using a pre-trained encoder-decoder network initially trained on a widely-used extensive dataset like ImageNet. Subsequently, they fine-tuned this network on the available cardiac data set for segmentation tasks. However, a significant challenge arises due to the considerable dissimilarity between the data characteristics used for pre-training and the specific nature of ultrasound cardiac images. These pre-trained networks are predominantly trained on vast collections of natural scene images, which starkly differ from the unique characteristics of cardiac ultrasound images. As a result, fine-tuning of pre-trained networks might not yield optimal results for cardiac ultrasound images. The inherent dissimilarity in the distribution and characteristics of the data between the pre-training data set and the ultrasound images can limit the effectiveness of fine-tuning efforts.

In view of the above problems associated with the state of the art, there is a need of a novel approach or process to mitigate the data scarcity problem in cardiac structure segmentation.

### OBJECTIVES OF THE INVENTION

The primary objective of the present invention is to facilitate the extraction of informative features tailored to the unique characteristics of cardiac images.

Another objective of the present invention is to provide for segmentation under limited number of annotated samples.

Other objects and advantages of the present invention will become apparent from the following description taken in connection with the accompanying drawings, wherein, by way of illustration and example, the aspects of the present invention are disclosed.

### SUMMARY OF THE INVENTION

The present invention provides an optimized class specific embedding generator and multi-branch decoder deep network for cardiac structure segmentation. The network comprises receiving labeled images and unlabeled echocardiogram image from an image/training dataset by an encoder network. The encoder network comprising a series of a double convolution layer followed by a max pooling layer to extract a plurality of feature maps using a training dataset. The encoder network is configured to extract image features from the labeled images and to map image features to multiple class specific feature. Further, the encoder network creates a prototype from the labeled images by computing average of generated embeddings. A splitter layer receives encoded features subtracted from the learned prototype, and the splitter layer is configured to transform the differential features into class specific embeddings. The splitter layer weights are learned during the training of encoder using the Matryoskha classifier. A decoder network receives class specific embeddings as input from the splitter layer, and the decoder network is configured to decode class specific features to reconstruct a segmentation map of cardiac structures from cardiac images.

### BRIEF DESCRIPTION OF DRAWINGS

The present invention will be better understood after reading the following detailed description of the presently preferred aspects thereof with reference to the appended drawings, in which the features, other aspects and advantages of certain exemplary embodiments of the invention will be more apparent from the accompanying drawing in which:

FIG. 1 illustrates a block diagram of multi-branch encoder-decoder deep network.

FIGS. 2(a)-(d) illustrate segmentation results obtained on patient 90 end diastole cardiac images from multi-branch encoder decoder deep network trained on 10% training data where (a) original image (b) annotation mask (c) predicted mask (d) Area overlap between the predicted mask and annotation mask.

FIGS. 3(a)-(d) illustrate segmentation results obtained on patient 90 end cardiac images from multi-branch encoder decoder deep network trained on 50% training data where (a) original image (b) annotation mask (c) predicted mask (d) Area overlap between the predicted mask and annotation mask.

FIGS. 4(a)-(d) illustrate segmentation results obtained on patient 111 end diastole cardiac images from the multi-branch encoder decoder deep network trained on 80% training data where (a) original image (b) annotation mask (c) predicted mask (d) Area overlap between the predicted mask and annotation mask.

FIGS. 5(a)-(d) illustrate segmentation results obtained on patient 234 end diastole cardiac images from multi-branch encoder decoder deep network trained on 10% training data where (a) original image (b) annotation mask (c) predicted mask (d) Area overlap between the predicted mask and annotation mask.

FIGS. 6(a)-(d) illustrate segmentation results obtained on patient 234 end diastole cardiac images from multi-branch encoder decoder deep network trained on 50% training data where (a) original image (b) annotation mask (c) predicted mask (d) Area overlap between the predicted mask and annotation mask.

FIGS. 7(a)-(d) illustrate segmentation results obtained on patient 234 end diastole cardiac images from multi-branch encoder decoder deep network trained on 80% training data where (a) original image (b) annotation mask (c) predicted mask (d) Area overlap between the predicted mask and annotation mask.

FIGS. 8(a)-(b) illustrate variation of the DICE coefficient with the increase in the training percentage of (a) end-diastole and (b) end-systole endocardial images.

FIGS. 9(a)-(b) illustrate variation of the DICE coefficient with the increase in the training percentage of (a) end-diastole and (b) end-systole epicardial images.

### DETAILED DESCRIPTION OF THE INVENTION

The following description describes various features and functions of the disclosed system with reference to the accompanying figures. In the figures, similar symbols identify similar components, unless context dictates otherwise. The illustrative aspects described herein are not meant to be limiting. It may be readily understood that certain aspects of the disclosed system can be arranged and combined in a wide variety of different configurations, all of which have not been contemplated herein.

Accordingly, those of ordinary skill in the art will recognize that various changes and modifications of the embodiments described herein can be made without departing from the scope of invention. In addition, descriptions of well-known functions and constructions are omitted for clarity and conciseness.

Features that are described and/or illustrated with respect to one embodiment may be used in the same way or in a similar way in one or more other embodiments and/or in combination with or instead of the features of the other embodiments.

The terms and words used in the following description are not limited to the bibliographical meanings, but, are merely used to enable a clear and consistent understanding of the invention. Accordingly, it should be apparent to those skilled in the art that the following description of exemplary embodiments of the present invention are provided for illustrative purpose only and not for the purpose of limiting the invention.

It is to be understood that the singular forms “a”, “an” and “the” include plural referents unless the context clearly dictates otherwise.

It should be emphasized that the term “comprises/comprising” when used in this specification is taken to specify the presence of stated features, integers, steps or components but does not preclude the presence or addition of one or more other features, steps, components or groups thereof. The equations used in the specification are only for computation purpose.

The present invention provides a teacher-student framework designed for semantic segmentation of cardiac ultrasound images by extracting cardiac structures from cardiac images using optimized class specific embedding generator and multi-branch encoder-decoder deep network.

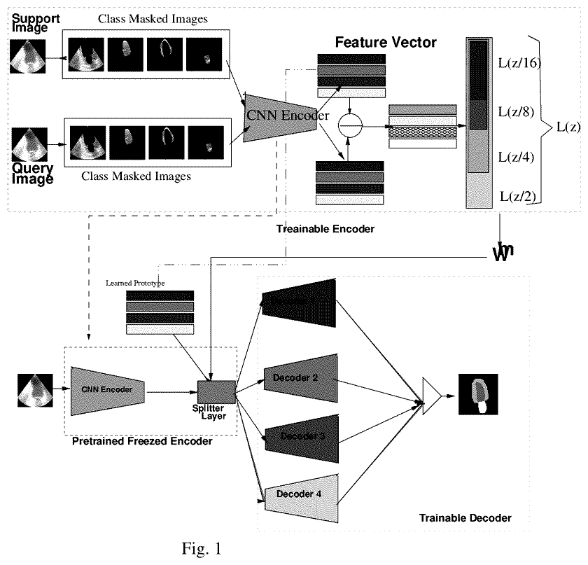

FIG. 1 illustrates a block diagram of a multi-branch encoder-decoder deep network. The multi-branch encoder-decoder deep network comprises an encoder neural network, a multi-branch decoder neural network, and a Matryoshka learning module.

In a preferred embodiment, a teacher-student framework is provided in the multi-branch encoder-decoder deep network. The teacher network consists of a series of encoder blocks followed by the fully connected Matryoshka linear layer. The student network, on the other hand, is constructed as an encoder-decoder network featuring a single encoder and multiple decoders. The teacher network compute the average of the support set embeddings that may be called a prototype, which is considered as a representation of the population. The prototype is subtracted from the query image embedding and is fed to the fully connected layer. The support set comprises of plurality of echocardiography images which acts as labeled dataset for the encoder network. The teacher network takes on the task of learning the distinctive embeddings of cardiac structures by using a set of support images for which annotated samples are accessible. The training of the teacher network follows a few-shot learning paradigm.

For example, an input image I may be fed into the teacher network, the output Eb which is a one-dimensional embedding vector may be computed by equation (1) as:

$$Eb = Maxpool ( ReLU ( {Cw}_{pq}^{{L}_{2}} \text{?} ReLU ( {Cw}_{pq}^{{L}_{1}} \text{?} I ) ) )$$

where, Cw^{L1} and Cw^{L2} represents the convolution kernels of layer L which is applied on pixel position (p,q). The encoded embedding is used to generate the prototype for each region.

The prototype for the region is learned during the training of the encoder. The average of the support set embeddings also called a prototype, which is considered a representation of the population. The support image embeddings for each class

$$( {Eb}_{iz}^{S} )$$

are averaged over a set of V images to obtain the prototype embedding

$${P}_{i = 1}^{C}$$

for each region c. Ine prototypes may be computed by equation (2) as:

$${P}_{i = 1}^{C} = \frac{1}{V} {\sum }_{z = 1}^{V} {Eb}_{iz}^{S}$$

In an exemplary embodiment, a support set of size V, only one query image is utilized in this case. The present invention may use few-shot learning framework to train the encoders in the teacher network. The training set may be divided into two parts: a support set denoted as S and a query set denoted as Q. The images in the support set and the query set are masked with their ground truth to generate the images of C classes. The differential characteristics of the query image are subsequently calculated by measuring the deviation from the prototype. The difference between the prototype P^{c} and the query Qⁱ for each class in C. Mathematically, it may be represented by equation (3) as:

$${MO}_{i = 1}^{C} = ( {P}^{i} - {Eb}^{{Q}_{i}} ) \qquad i = 1 , 2 , 3 , \cdot C ,$$

where MOᵢ is the deviation between the two embeddings, Pⁱ represents the prototype for class C and Eb^{(Q}^{ᵢ}⁾ represents the image embedding for the i^{th} region of query image. The obtained MOᵢ is fed into the Matryoshka representation layer to learn the differential embeddings of the class.

The training of the teacher network is carried out using a stochastic gradient algorithm. A loss function for the teacher network is considered to be cross-entropy, as the loss function has been shown to perform well in error estimation in multiclass problems. The cross-entropy may be computed by equation (4) as:

$$H ( p , q ) = - {\sum }_{x ∈ X} p ( x ) log q ( x )$$

where x is the input to the system and p(x) is probability distribution of the system and q(x) is the actual distribution. Here, the actual class is computed by setting the index of the label values present in the ground-truth image.

The splitter layer serves the role of mapping the differential embeddings to class-specific embeddings. It should be noted here that the input to the splitter layer is the difference between the prototype and generated embedding. The weights of the splitter layer are obtained from the Matryoshka representation and may be computed by equation (5) as:

$$W * [ \cdot , i ] = max \qquad \begin{matrix}M \\ \{ ( A {\{}_{j} , \cdot \} )\end{matrix} σ ( {A}_{i , j}^{m} \times x ) , \qquad i = 1 , 2 , … C , j = 1 , 2 , … d$$

where, m is the dimension considered out of the embeddings of M dimensional, A is the weight matrix connection the features and the classes and C represents the classes that are learned during the training of the encoder. The weight matrix is computed after the completion of the training of the encoding is passed as input to the splitter layer. The input to the splitter layer is the computed embedding from the image fed into the student network, the weight matrix W, and the prototype learned during training of the teacher network. The embedding is subtracted from the prototype and the resultant is multiplied with each column of the weight matrix W. The multiplied vector is fetched into the decoder for decoding.

In an embodiment, the student network may be trained with a backpropagation algorithm with an Adaptive Moment Estimation (ADAM) optimizer [13, 5]. The rationale for considering the ADAM optimizer is that it allows for an adaptive learning rate for each parameter, which leads to faster convergence. ADAM is an extension of the stochastic gradient descent (SGD) optimization algorithm that computes individual adaptive learning rates for different parameters. ADAM combines ideas from RMSprop (Root Mean Square Propagation) and Momentum optimization techniques. ADAM adapts the learning rates for each parameter based on the estimates of the first and second moments of the gradients, which helps in training models more efficiently, especially in scenarios with sparse gradients or noisy data. In one case, binary cross-entropy loss [5, 19] is utilized for training the network. Binary Cross-Entropy Loss, often referred as “BCE loss” or “logistic loss” is a loss function commonly used in binary classification tasks in machine learning. It measures the difference between two probability distributions: the actual distribution of the labels and the predicted probability distribution given by the model. The loss function may be calculated by equation (6) as:

$$BCE ( y , \hat{y} ) = - \frac{1}{N} {\sum }_{i = 1}^{N} [ {y}_{i} log ( \hat{{y}_{1}} ) + ( 1 - {y}_{i} ) log ( 1 - \hat{{y}_{1}} ) ]$$

where y represents the ground truth mask, ŷ represents the predicted mask, and N is the number of pixels in the images. The BCE loss penalizes models more heavily for confidently incorrect predictions, i.e., when the predicted probability diverges significantly from the true label. It is particularly well-suited for binary classification problems and is commonly used as the loss function when training models with sigmoid activation functions in the output layer. It may be noted that the encoder layer and splitter layer parameters are frozen in the student network, and only the decoder layer parameters are updated during training. This is done to ensure that the learned encoders do not suffer catastrophic forgetting of learned differential features.

According to FIG. 1, a high level encoder neural network is provided. The encoder network is configured to train on a training set. The training set may either be internally stored in a storage means of a computing device or provided as an input via an external storage means. The encoder block consists of a double convolution layer followed by a batch normalization layer and a ReLU activation function.

The encoder network receives labeled and unlabeled echocardiogram images from an image or training dataset. The encoder network comprising of a series of double convolution layer followed by a max pooling layer, extract a plurality of feature maps using a training dataset. In an embodiment, the network is trained using a K-way few-shot learning framework. The convolution and pooling operations may be applied on the images sequentially in each layer of encoder network to achieve encoding. After pooling the ReLU activation is applied to pass on the positive valued computed features to the next layer of the encoder. In the last layer of encoder network a sigmoid function is applied to obtain the probability value that represents the encoding.

The encoding may be computed in a support set and one or more query images. The support set images may be split into k region images by masking them with their ground-truth images, where the k regions are considered k classes. The trained encoder network, the prototype, and the weights of the Matryoshka network are frozen and used in a student network. During training, the encoder network is configured to extract image features from the labeled images and image features are mapped to multiple class specific features. Further, the class specific features are extracted from the labeled images and average of labeled embeddings are computed. In an embodiment, the encoder network may be configured using a convolutional neural network (CNN) model.

According to FIG. 1, a multi-branch decoder is provided. The multi-branch decoder is configured to generate the segmentation of each region of the cardiac images. The decoder network implicitly transforms the segmentation task to multitask learning problem. The multi-branch decoder comprises of plurality of decoders. The decoder is configured to share same differential features into class-specific embeddings to reconstruct the segmentation map of cardiac structures. The differential features is the difference between prototype and encoded features obtained from the encoder.

The disclosed multi-branch decoder is distinct from the traditional U-Net architecture. The decoders share the same skip connection from the encoders, and this distinguishes the approach of the present invention from the conventional U-Net architecture. The motivation for employing multiple decoders lies in the variable convergence times required for different regions within the input. This discrepancy arises from variations in the size and shape of these regions. The experimental results demonstrated that convex regions exhibit faster convergence compared to non-convex regions. Consequently, approach of the present invention assigns individual decoders distinct learning durations, effectively allowing them to adapt to varying convergence rates. In addition, a splitter layer is introduced that serves the role of mapping the differential embeddings to class-specific embeddings. The input to the splitter layer is the difference between the prototype and generated embedding, and may be computed using Equation (3). This strategic inclusion enables to reframe the problem as a multitask learning scenario. In the present invention, each decoder is responsible for generating a specific class output. This strategy not only takes advantage of collective learning but also utilizes the network's capacity to excel in distinct tasks. The weights of the splitter layer may be calculated from the Matryoshka representation by Equation (5).

The multiple tasks of segmentation can be carried out by each of the decoders individually and can be considered independent. Hence, a generated embedding from the encoder is representation is transformed into multiple classes out at the splitter layer as [symbol] _{i=12,3, . . . , c}=Eb·Wᵢ where Eb of dimension 1×D is the embeddings obtained from the encoder and Eb is the embeddings obtained after the transformation. It may be observed that W is of dimension d×C for d∈D, whereas d may be tracked for each of the classes and based thereon the d dimension of the embedding is selected for the dot product with weights to ensure the dimensionality constraint.

The obtained [symbol] is sent to each of the decoders to generate a segmentation map (SMAP) for each of the classes. The final layer of the decoder has a sigmoid layer. The final combined output segmentation map ŷ may be computed as: ŷ=max_{i=1, . . . , c} SMAPᵢ.

In accordance with FIGS. 3(a)-(d), 4(a)-(d), 5(a)-(d), 6(a)-(d), 7(a)-(d), 8(a)-(b) and 9(a)-(d) and disclosure of the present invention, an exemplary illustration is provided, wherein an experimental setup including measures used for comparison and results are described. It may be appreciated that said exemplary illustration should not be construed to limit the scope of the present invention.

Experiment: In an experimental setup, the experiment was performed on an instance of A100 GPU DGX system with 80 GB Ram and 4 central processing unit (CPU) cores, 40 GB GPU and Linux operating systems. The disclosed features and method thereof was programmed using a Python programming platform. The experiment was done in the CAMUS dataset comprising of 1000 images captured during the end diastole and end systole phase of cardiac cycles.

Dataset: In the experiment conducted, the CAMUS dataset was utilized. The data set comprises 1000 2-chambered 2D ultrasound images, with 50% of the images exhibiting ejection fractions (EF) below 45%, indicating pathological risk. Additionally, 19% of the images have a low signal-to-noise ratio. Ground truth annotations are provided for the endocardium, myocardium, and atrium of 2D ultrasound images in both end-diastole and end-systole phases. During the training phase, 50% of the end-diastole images were sampled for training purposes. All images are resized to dimensions of 128×128 and normalized within the range of [0, 1]. Using the ground-truth information supplied, the endocardium, myocardium, and atrium regions extracted from the images to create the respective support set classes.

Metrics: DICE Index (DI) [12, 2] is the statistical tool to measure the similarity between the segmentation map obtained X and the ground truth provided G, and computed as

$$Dice Index = \frac{2 \times \vert X ⋂ G \vert}{\vert X \vert + \vert G \vert} .$$

The DICE value is in the range of [0, 1]. The higher the value, the better the performance of the technique. The other metric used for comparison is the Hausdorff distance (HD). The distance quantifies the maximum distance between the corresponding points in the segmented and reference objects. High values of the Hausdorff distance indicate significant discrepancies between segmentation and ground truth, highlighting regions where the algorithm may struggle. On the contrary, lower values signify a closer match between the two sets of points and thus a more accurate segmentation. It may be computed as

$${d}_{H} ( X , G ) = max \{ {inf}_{g ∈ G} d ( x , g ) \vert x ∈ X \} .$$

Hyper-parameters: The hyper-parameters of the teacher network are the number of convolution layers, the size of the convolutional mask, the Matryoshka encodings, and the learning rate of the network. It was experimentally observed that 7 encoding layers with the number of filters in each layer [1, 16, 64, 128, 256, 512, 1024] provide good results during training. In one case, a 1024-dimensional feature representation for the embeddings, as it was found to be enough for the presently used set of images. However, it can vary with the problem in question. In another case, 6 Matryoshka encodings are considered with sizes of [64, 128, 256, 512, 1024]. The encoding with sizes can be varied and fine-tuned. During the experiment, a level of encoding [128, 512] was observed to be necessary to obtain a good representation for ultrasound images. The learning rate for the teacher network was set at 0.0001 because the network should learn the details of differential characteristics at different granularities properly. Thus, a low learning rate would help improve learning characteristics.

The hyper-parameter of the student network is the learning rate which is set to 0.001 which provides a significant result. It should be noted here that the same encoding layer of the teacher network is used in the student encoder-decoder network. Hence, the size of the encoder and decoder is kept the same as the size of the encoder and the network of the teacher. The network was trained until the training error reduces to 1×10⁻².

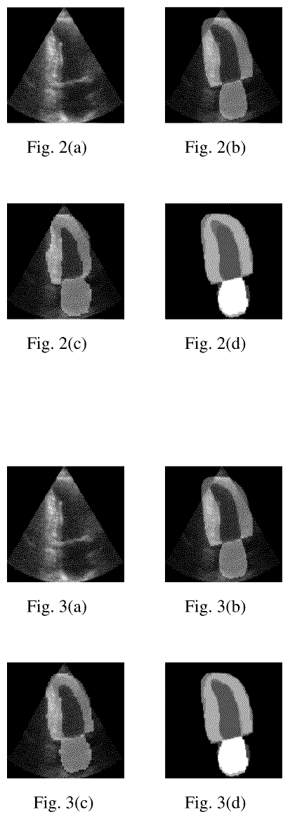

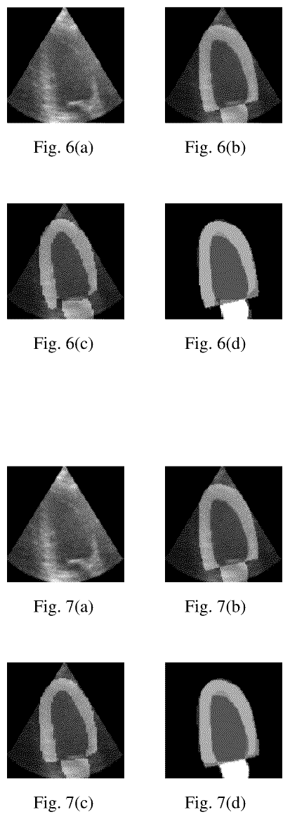

Result and Analysis: The experiment is carried out on 1000 two-chamber cardiac images obtained at the end-systole and end-diastole phases of the cardiac cycle. Evaluation of the proposed approach was performed using two quantitative metrics, namely, the DICE coefficient and the Hausdroff distance, and the quantitative biological marker, i.e., the ejection fraction and LV volume. The qualitative results of the samples obtained from the proposed approach along with the ground truth images for the two phases are shown in FIGS. 2-7. It can be observed that the results are shown for three different training sizes (i.e., 10%, 50%, and 90%). In addition, three colors, namely green, red, and blue, are used to represent the epicardium & endocardium of the left ventricle and atrium respectively. On analyzing the results obtained from the proposed approach, it is observed that the overlap between the ground truth and the predicted mask is high for the network trained with 90% training data. However, as per observation that the multichannel decoder network trained with the other two percentages of data performs significantly well and the deviation in the test results of the ground-truth images is low as shown in FIGS. 3(a)-(d), 4(a)-(d), 6(a)-(d), and 7(a)-(d). Thus, the qualitative results support the hypothesis that the proposed approach can perform robustly with limited training samples. The decoder network receives class specific embeddings as input from the splitter layer, the decoder network comprises of a plurality of decoders. The decoder network configured to decode class specific features to reconstruct a segmentation map of cardiac structures from cardiac images.

The quantitative results of the present approach and other baseline approaches are provided in the following Tables 1-3.

Table 1 illustrates the performance evaluation on the test set of left ventricular endocardium (LV endo) segmentation, with metrics the Dice Index (DI) and mean Hausdorff distance (HD).

TABLE 1

| | |
|---|---|
| End Diastole | End Systole |

metrics

| Aglorithms | DI | HD | DI | HD |
|---|---|---|---|---|
| Segnet | 0.890 | 6.7 | 0.899 | 5.3 |
| U-net + RB | 0.894 | 6.1 | 0.892 | 5.1 |
| U-net + syndata | 0.925 | 4.9 | 0.901 | 5.2 |
| GUDU + U-net | 0.946 | 4.7 | 0.929 | 4.7 |
| Proposed | 0.932 | 4.5 | 0.911 | 4.1 |

Table 2 illustrates performance evaluation on the test set of left ventricular epicardium (LV epi) segmentation, with metrics the Dice Index (DI) and mean Hausdorff distance (HD).

TABLE 2

| | |
|---|---|
| End Diastole | End Systole |

metrics

| Aglorithms | DI | HD | DSC | HD |
|---|---|---|---|---|
| Segnet | 0.902 | 6.7 | 0.899 | 5.3 |
| U-net + RB | 0.894 | 6.1 | 0.892 | 5.1 |
| U-net + syndata | 0.925 | 4.9 | 0.901 | 5.2 |
| GUDU + U-net | 0.946 | 4.7 | 0.929 | 4.7 |
| Proposed | 0.932 | 4.5 | 0.911 | 4.1 |

Table 3 illustrates performance evaluation on the test set of left atrium (LA) segmentation, with metrics the Dice Index (DI) and mean Hausdorff distance (HD).

TABLE 3

| | |
|---|---|
| End Diastole | End Systole |

metrics

| Aglorithms | DI | HD | DI | HD |
|---|---|---|---|---|
| Segnet | 0.821 | 7.2 | 0.843 | 6.7 |
| Unet + RB | 0.848 | 6.8 | 0.888 | 6.4 |
| Unet + syndata | 0.890 | 5.5 | 0.910 | 5.9 |
| GUDU + Unet | 0.894 | 5.4 | 0.926 | 5.6 |
| Proposed | 0.901 | 4.9 | 0.912 | 5.7 |

Tables 1-3 presents the DICE index (DI) and Hausdroff distance (HD) obtained from the ground-truth images and the predicted mask obtained from the different approaches. It is evident that the proposed approach and U-net GUDU perform best compared to the other approaches in both phases of the cardiac cycle. However, the proposed approach provides competitive results with limited training data and low-dimensional embeddings, compared to GUDU with Unet where the data set is enriched with data augmentation. Observing the HD score obtained for the proposed approach, it is evident that the proposed approach has the least deviation in the structures. This may be attributed to the differential characteristics learned by the teacher network when comparing with the exemplar embeddings constructed from the set of support images and the decomposition of the reconstruction problem by the decoder to a multitask learning problem.

Clinical parameters such as the ejection fraction and LV volume at the end diastole and the end-systole of the cardiac cycle are used in during the experiment. The correlation values of the calculated parameters with the ground truth ones are provided in Table 4. It is evident that the correlation is high between both phases, which highlights the efficacy of the proposed approach in parsing the cardiac structure.

Table 4 illustrates the clinical evaluation on the test set of left Ventricle segmentation, with metrics Ejection Fraction (EF), and LV volume at end-diastole and systole image of cardiac structure.

TABLE 4

metrics

| | Correlation | End Diastole | End Systole |
|---|---|---|---|
| Aglorithms | EF | LV volume | LV volume |
| Segnet | 0.792 | 0.963 | 0.972 |
| Unet + RB | 0.796 | 0.978 | 0.965 |
| Unet + syndata | 0.864 | 0.979 | 0.974 |
| GUDU + Unet | 0.897 | 0.977 | 0.981 |
| Proposed | 0.901 | 0.981 | 0.983 |

The network performance is studied by varying the size of the training and compared the results of the proposed approach with two of the baseline approaches, namely Segnet and UNet with residual block. It may be noted that other two approaches are not discussed here as both approaches increase the training size with data augmentation, which may hinder understanding the performance of the data with limited training samples.

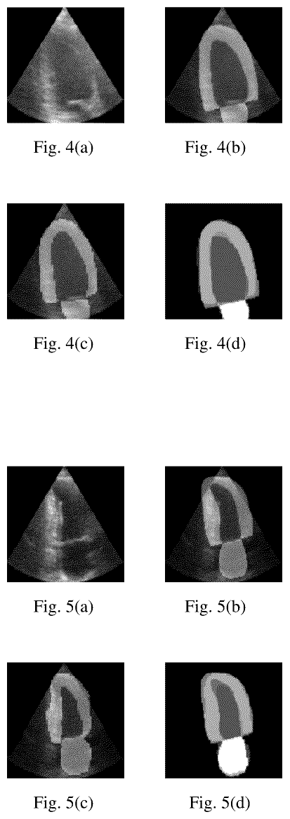

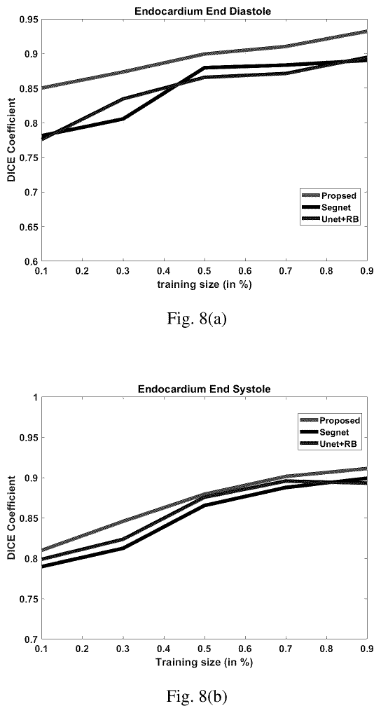

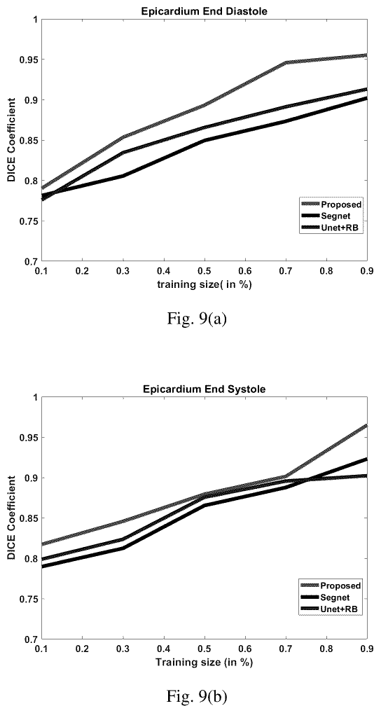

The results of the DICE index for the endocardium and epicardium are provided in FIGS. 8(a)-(b) and 9(A)-(b). It is evident that the performance of the proposed approach is not much affected by the size of the training, and with limited training data, the proposed approach can achieve significant results compared to the other two approaches. Moreover, the performance of the network increases logarithmically with increasing training size, as is evident from the figures. During training the teach network, it was also observed that the complexity of the shape of the regions determines the size of the embeddings. With 128-512-sized embeddings, we were able to reconstruct the endocardium and atrium as opposed to the epicardium, which required 512-sized embedding. However, it was observed that with the use of lower-size embedding, there can be slight deviation in orientations, as visible in FIGS. 3(a)-6(d). In contrast, with a larger training set, this can be compensated, as visible in FIGS. 4(a)-7(d). As is evident from Table 4, the correlation values are high between the ejection fraction and the LV volumes. Hence, it can be inferred that despite the deviation in orientation the structure of the ventricle is well preserved.

In an embodiment, a method for cardiac structure segmentation using an optimized class specific embedding generator and a multi-branch decoder deep network is provided. The method comprising the steps of: receiving labeled images and unlabeled echocardiogram image from an image training dataset by an encoder network, wherein the encoder network comprising a series of a double convolution layer followed by a max pooling layer to extract a plurality of feature maps using a training dataset. The encoder network is configured to: extract image features from the images; learn the weights matrix of splitter layer to transform class specific features from the generated image features using matryoshka learning; and compute average of labeled image embeddings to form the prototypes. The method further comprises the step of receiving encoded class specific features subtracted from the learned prototype by a splitter layer, wherein the splitter layer is configured to transform the differential features into class specific embeddings. The method further comprises the step of receiving a class specific embeddings as input from the splitter layer by a decoder network, wherein the decoder network is configured to decode class specific features to reconstruct a segmentation map of cardiac structures from cardiac images. The decoder network comprises a plurality of decoders.

In an embodiment, a computer program product comprising computer-readable instructions for implementing a method for cardiac structure segmentation using an optimized class specific embedding generator and a multi-branch decoder deep network on a computer or a processing device is provided.

While this invention has been described in connection with what is presently considered to be the most practical and preferred embodiment, it is to be understood that the invention is not limited to the disclosed embodiments, but, on the contrary, is intended to cover various modifications and equivalent arrangements included within the scope of the appended claims.

We claim:

1. A method for cardiac structure segmentation using an optimized class specific embedding generator and a multi-branch decoder deep network, comprising:
a) receiving labeled images and unlabeled echocardiogram image from an image training dataset by an encoder network, wherein the encoder network comprises a series of a double convolution layer followed by a max pooling layer;
b) extracting a plurality of feature maps using the training dataset by the encoder network, wherein the encoder network is configured to:
i. extract image features from the images;
ii. learn the weights matrix of splitter layer to transform class specific features from the generated image features using matryoshka learning; and
iii. compute average of labeled image embeddings to form the prototypes;
c) receiving one or more encoded class specific features subtracted from the learned prototype by a splitter layer, wherein the splitter layer is configured to transform the differential features into class specific embeddings; and
d) receiving class specific embeddings by a decoder network as an input from the splitter layer, wherein the decoder network is configured to decode class specific features to reconstruct a segmentation map of cardiac structures from cardiac images.

2. The method as claimed in claim 1, wherein the double convolution layer provides a non-linear feature encoding.

3. The method as claimed in claim 1, wherein the max pooling layer reduces the size feature mapping by removing the redundant information.

4. The method as claimed in claim 1, wherein the encoder network is using a convolutional neural network (CNN) model.

5. The method as claimed in claim 1, wherein the splitter layer maps the image encoding to a class specific encoding.

6. A computer program product comprising computer-readable instructions for implementing the method of claim 1 on a computer or a processing device.
